(19) United States
(12) Patent Application Publication
(10) Pub. No.: US 2013/0164810 A1
(43) Pub. Date: Jun. 27, 2013

(54) SULFIDE GENERATION VIA BIOLOGICAL REDUCTION OF DIVALENT,TETRAVALENT OR PENTAVALENT SULFUR CONTAINING COMBUSTION FLUE GAS OR LIQUOR

(71) Applicants: Douglas Warkentin, Vancouver (CA); Norman Chow, Vancouver (CA)

(72) Inventors: Douglas Warkentin, Vancouver (CA); Norman Chow, Vancouver (CA)

(73) Assignee: KEMETCO RESEARCH INC., Richmond (CA)

(21) Appl. No.: 13/537,529

(22) Filed: Jun. 29, 2012

Related U.S. Application Data
(60) Provisional application No. 61/502,424, filed on Jun. 29, 2011.

(51) Int. Cl. C12P 3/00

(52) U.S. Cl. CPC C12P 3/00
USPC 435/168

(57) ABSTRACT

The present invention relates to the biologically catalyzed, anaerobic generation of sulfide species as sulphide, hydrosulfide or hydrogen sulfide in anaerobic bioreactors from the reduction of tetravalent sulfur derived from one or more sources including sulfur dioxide containing combustion flue gas, or the reduction of divalent or pentavalent sulfur containing liquors such as thiosulfate or dithionate containing liquors. Flue gas sources of sulfur dioxide also contain one or more bio-nutrients or energy sources. The generated sulfide is useful for numerous applications including waste treatment and metals recovery as sulfides.

### PRIOR APPLICATION

This non-provisional application claims the priority of prior U.S. provisional application No. 61/502,424, filed Jun. 29, 2011.

### TECHNICAL FIELD

The invention relates to the field of biologically catalyzed reduction of tetravalent sulfur compounds, derived from sulfur dioxide containing flue gas, or divalent sulfur containing process liquors, such as thiosulfate containing liquors, or pentavalent sulfur containing liquors, such as dithionate containing liquors, where such reduction results in the creation of sulfide species as sulfide, hydrosulfide or hydrogen sulfide. Sulfide species can be used either for the removal of metals from solution, as an intermediate in the removal of sulfur compounds from the solution, or both applications.

### BACKGROUND OF THE INVENTION

U.S. Pat. No. 5,196,176 to Buisman describes the desulphurization of sulfur dioxide containing flue gas, e.g. from oil-fired or coal-fired power stations via 1) sulfur dioxide scrubbing as alkaline aqueous sulfite 2) anaerobic bacterial conversion of sulfite to sulfide 3) aerobic bacterial conversion of sulfide to sulfur. (It describes CO and H₂ as bioreactor nutrients column 2, lines 55-56). This invention does not utilize the waste treatment or metal recovery value of sulfide by converting it all to elemental sulfur.

U.S. Pat. No. 5,587,079 to Warkentin et al describes the bio-reduction of sulfate to hydrogen sulfide with anaerobic bacteria wherein a portion of bacteria nutrients including CO and H₂ are derived from partial oxidation of a carbon containing fuel. This patent does not describe the use of sulfur dioxide containing flue gas for use in bio-reduction of sulfite to sulfide species including hydrogen sulfide. Using sulfite as the sulfur source means that the stoichiometric energy requirement for reduction is only 75% of that required for sulfate. This arises from the H₂ requirement for autotrophic reduction as indicated by the net reduction reactions:

4H₂+H₂SO₄→H₂S+4H₂O

and:

3H₂+H₂SO₃→H₂S+3H₂O

The relative energy requirement for the reactions can also be determined thermodynamically. The equilibrium aqueous half cell reaction for reduction of sulfite to hydrogen sulfide proceeds according to the following reaction:

SO₃²⁻+5H₂O+6e−→H₂S+8OH⁻

The standard free energy of reaction, using available data (Pourbaix, 1966, pg 98, 546) for this reaction is calculated to be 92,250 calories/mole.

The equilibrium aqueous half cell reaction for reduction of sulfite to hydrogen sulfide proceeds according to the following reaction:

SO₄²⁻+6H₂O+8e⁻→H₂S+10OH⁻

The standard free energy of reaction, using available data (Pourbaix, 1966, pg 98, 546) for this reaction is calculated to be 134,990 calories/mole. This calculation results in an even lower relative energy requirement, at 68.3% of that required for sulfate.

Numerous researchers have described processes for treating mine drainage, waste gypsum, flue gas desulfurization wastes and other metal- and/or sulfur-containing wastes using cultures of sulfate reducing bacteria (SRB) both in controlled bioreactors and in constructed wetlands. In a few cases this has included separation of the metal precipitation and the sulfide generation, and a small number of commercial operations have been reported in the literature using biogenic sulfide to recover at least one valuable metal from a waste stream.

Some researchers describe the biological reduction of wastes from power plant or smelter stack gases. In most cases these processes incorporate substantially different biological systems than the current invention, including the use of aerobic or anaerobic systems with organic nutrients. In the case of anaerobic systems, additional stages are required to collect and concentrate the sulfur compounds prior to feeding to the bioreactor, primarily to exclude oxygen, which is generally present in substantial quantities in these gas streams. In general, combustion processes that result in waste gas streams containing sulfur dioxide are operated with a high level of excess oxygen to prevent the formation of products of incomplete oxidation, such as carbon monoxide. The resulting gas stream will also contain excess oxygen and a significant part of the sulfur will become further oxidized to sulfur trioxide, which becomes sulfuric acid or sulfate when dissolved into solution. Such a stream cannot be fed directly to an anaerobic reactor without severely limiting its operating effectiveness due to the combination of oxygenation and acidification of the solution. Proposed systems generally therefore include a separate scrubbing stage to collect the sulfur dioxide and trioxide into an alkaline solution or an organic collector and thus separate it from the oxygen-containing gas stream, allowing the resulting waste solution containing sulfite and sulfate to then be fed to the bioreactor. This process itself is also likely to have the effect of converting much of the sulfite to sulfate. Descriptions of systems of this type that have been identified in previous patents, or in the literature, invariably describe a process intended for the treatment of waste off-gas streams. Generally the end product is elemental sulfur generated through the oxidation of the resulting dissolved sulfide ions or hydrogen sulfide off-gas.

When these gas streams are scrubbed into a solution, the resulting solution is likely to have a low sulfite concentration and be highly oxygenated; making it of limited use as a bioreactor feed solution. The current invention differs in that the combustion of the sulfur-containing fuel is internal to the process, and carried out under controlled conditions designed to produce a gas stream suitable for generating a partially reduced sulfur stream as feed to the bioreactor. Of particular importance is the limiting of oxygen feed to the combustion stage, with the result that off-gas streams are highly depleted in oxygen. Any other incomplete oxidation products that may result, such as carbon monoxide, can also be taken up and utilized in the bioreactor.

### Bioreactor Operation

Biologically catalyzed generation of sulfide is an important naturally occurring phenomenon, which in nature acts as a key component in the sulfur cycle. A wide variety of bacteria and other microorganisms have evolved which can utilize sulfate or other sulfur species as a terminal electron acceptor under anaerobic and anoxic conditions. These microorganisms can function in many different environments using a number of different possible substrates that utilize different metabolic cycles. In most environments these reactions will function as part of a mixed population of both competing and complimentary microorganisms (Madigan et al 2003, Muyzer et al 2008).

In recent years numerous researchers have developed technologies to make use of these naturally occurring organisms under controlled conditions. Applications have generally been aimed at waste water treatment, with some elements of recovery of dissolved metals from these waste streams (e.g. U.S. Pat. Nos. 4,839,052; 5,196,176; 5,587,079; 6,852,305). To date commercialization of these technologies has been slow, due primarily to the relatively high costs. Operating costs in particular associated with the required supply of energy to the bacteria are an important consideration.

The current invention therefore is primarily concerned with multiple and unexpected operational benefits that are derived from a novel combination of the choice of bioreactor operating parameters and the methods of supplying sulfur and energy to the bioreactor. In most of the reported work in this field, sulfur is provided to the bioreactor in the form of sulfate dissolved in a feed solution as a metal sulfate, sulfate salt or dilute sulfuric acid. In this system the net reduction reaction in its most basic form is:

4H₂+H₂SO₄→H₂S+4H₂O

Depending on the substrate chosen, the energy required to drive this reaction may be provided by an organic acid such as lactate or acetate, an alcohol such as ethanol, or directly by hydrogen gas dissolved in solution. When using hydrogen as the principal energy source carbon dioxide is also required which is converted to new cell material during bacterial growth.

The use of hydrogen and carbon dioxide allows energy to be derived from inexpensive fuels via low-oxygen combustion or steam reforming to produce a gas stream rich in hydrogen and carbon dioxide, but rates of biological activity tend to be lower than with the use of organic energy sources.

With the current invention the principal sulfur source is not hexavalent sulfate, but a partially reduced divalent, tetravalent or pentavalent sulfur compound such as sulfite, thiosulfate or dithionate. The preferred embodiment would utilize sulfite derived from sulfur dioxide gas generated by combustion of sulfur-containing fuels under conditions chosen to derive the maximum process benefit. The sulfite reduction reaction requires less energy than sulfate, as indicated by:

3H₂+H₂SO₃→H₂S+3H₂O

In addition, because sulfate is the most stable species it must be activated by a separate enzyme for reduction to sulfite (Madigan et al 2003). This leads to the potential for much higher rates of reduction to H₂S from sulfite, making the use of inorganic gases as the carbon and energy sources more practical for large scale commercial application.

Previous work has included sulfite reduction in the context of the treatment of wastes from flue gas desulfurization at smelters or power plants (U.S. Pat. Nos. 4,614,588; 4,789,478; 5,269,929; 5,354,545; 5,976,868; and Scheeren et al 1992). In this previous work sulfite is a contaminant to be removed rather than a feed stock, and these processes do not allow for control of the combustion processes that generate the waste stream. In past work the oxygen content in the gas stream to be treated would be as high as 5% (Oilgae 2009) which would negatively affect bioreactor performance if added directly. Scrubber solution is therefore treated, which would commonly have a much higher conversion of sulfite to sulfate than the gas stream, especially when alkaline scrubbing is carried out. With the current invention, selection and control of fuels and burner operation result in unexpected overall process benefits.

### Reduced Sulfur Compounds

The operation of burners using precipitated metal sulfides and sulfur as a principal fuel results in the potential for creating closed-loop processes for sulfur. Controlling the burner operation to limit excess oxygen allows control of off-gas quality making it possible to feed the gas directly back to the bioreactor. Burning precipitates to remove sulfur and convert metals to oxides can serve as an important step in upgrading precipitated concentrates, but it also allows the release of a significant part of the energy that was supplied to the bioreactor to generate the sulfide reagent. Where required, this energy is readily applied to the warming of the bioreactor with the hot gas stream.

As is apparent from the stoichiometry, reduced sulfur compounds take less energy to convert to sulfide, so preventing unnecessary conversion of sulfite or other compounds to sulfate has a direct process and economic benefit. Also, most sulfide precipitates contain enough energy to sustain combustion, making a sulfide burner a suitable unit for combustion of bioreactor off-gas streams that have been stripped of their hydrogen sulfide. These gas steams will contain fuel values in the form of unused hydrogen and carbon monoxide and may also contain methane generated by competing methanogens in the bioreactor. These fuel values may be too dilute to be efficiently burned for energy recovery on their own but can be burned in the high temperature environment of the sulfide burner to recover energy and clean the gas prior to discharge or reuse.

### Sulfite Reduction Energy Sources

The principal energy sources for sulfite reduction in the bioreactor are the gases hydrogen and carbon monoxide. These can both be generated from low cost carbon-cased fuels through partial oxidation or gasification systems. Both hydrogen and carbon monoxide can be utilized in the bioreactor das an energy source for the reduction of sulfite. Hydrogen is preferred, as it can be directly utilized in sulfite reduction (Muyzer et al 2008). Carbon monoxide is used more slowly, and may need to first be converted to hydrogen and carbon dioxide by other bacteria, using the water shift reaction. This reaction can also be carried out in the combustion stage with steam and a catalyst to maximize the amount of hydrogen reaching the bioreactor:

CO(g)+H₂O(g)→H₂(g)+CO₂(g)

Carbon monoxide does have other potential benefits as an energy source as it has a higher solubility in water, which may result in improved mass transfer characteristics. Also, since carbon monoxide is converted via the reaction above to hydrogen and carbon dioxide, its presence will tend to have a stabilizing effect on solution pH.

### Carbon Dioxide

Carbon dioxide is required in the bioreactor as the source for carbon in cell growth. This is a small demand in comparison with its pH regulating function in the bioreactor. As shown in the generalized reduction reaction, sulfite reduction also generates alkalinity as a by-product, causing the pH to rise:

3H₂(aq)+SO₃²⁻(aq)→H₂S(aq)+H₂O(l)+2OH⁻(aq)

Carbon dioxide regulates the pH according to the reaction:

OH⁻(aq)+CO₂(g)→HCO₃⁻.(aq)

Excessive levels of carbon dioxide can, however, lower the solution pH below optimal levels and allow the growth of competing organisms to expand. It is therefore important to have the ability to control the addition rate of carbon dioxide. This is an important function of control of the operation of a partial oxidation burner or gasifier in the process. The preferred mechanism of control is to operate the combustion system with sufficient oxygen to ensure the generation of necessary carbon dioxide in the feed gas stream. The relative quantity of carbon dioxide to be generated can therefore be controlled through the adjustment of the air:fuel ratio in the burner.

### NOx

High temperature combustion in air often results in the production of small quantities of oxides of nitrogen, or NOx. These include primarily nitrous oxide, nitric oxide and nitrogen dioxide, with the first two being the most important. These compounds are soluble in water and form nitrite and nitrate. In large concentrations nitrate can inhibit sulfite reduction and lead to competition for available energy and nutrients, but in the concentrations produced in these combustion processes they can be utilized as nutrients, potentially reducing the requirement for feeding ammonium salts to the bioreactor as a nitrogen source.

In solution, nitrates are reduced to nitrite which certain sulfate reducing bacteria are capable of further reducing to ammonia:

NO₂⁻(aq)+7H⁺(aq)+6e⁻→NH₃(aq)+2H₂O(l)

In normal burner operation the NOx produced may not be sufficient to meet all of the nitrogen needs of the bioreactor, but for fuels such as coal this reaction could account for a quarter of the necessary nitrogen, while effectively dealing with these pollutants. (Greene et al, 2003; Moura et al, 2007; He et al, 2010).

### Carbonyl Sulfide

This compound is commonly present in small quantities in coal plant emissions and may increase in partial oxidation applications. This and other similar highly reduced sulfur compounds would represent a more direct supplementary source of sulfur for the process. Under alkaline conditions this compound can break down to carbon dioxide and sulfide without the need for biological activity (Svoronos et al, 2002).

### Fly Ash

Although not significant with all fuels, certain fuels such as coal, coke or biomass will generate a significant fly ash fraction when burned. This material generally has high alkalinity and can be used in acidic waste treatment applications as a supplementary source of alkalinity to the process. Depending on the fuel, these ash products may also be high in potassium and calcium, which are both added to the bioreactor as micronutrients. Use of fly ash would reduce reagent requirements while avoiding the need for separate collection and disposal (EUBIA 2007).

### Existing Technologies—Sulfide Precipitation

The principal purpose of the current invention is the generation of sulfide at a project site as part of a water treatment, metal recovery, or other industrial process. In current practise sulfide precipitation is a relatively uncommon choice for these applications, as available sulfide generation methods are costly relative to the most common water treatment and metal recovery alternatives.

### Biogenic Sulfide Systems:

Previous work has described a range of process configurations, including multi-stage sequential sulfide precipitation using biogenic hydrogen sulfide brought from a separate bioreactor in a carrier gas stream (Rowley 5,587,079). Biological systems for generating sulfide have been described using a range of bioreactor types, normally fixed-film or packed-bed bioreactors, with some incorporating biomass settling and recycle from the discharge. Most commonly the bioreactor utilizes a mixed population of SRB, although occasionally particular strains are specified. Also, most commonly the bioreactor operates by reducing sulfate, using an organic nutrient such as ethanol or lactate as the principal carbon and energy source. Use of reagent elemental sulfur or sulfur compounds resulting from flue gas desulfurization as the sulfur source have also been described, and the use of inorganic gaseous carbon and energy sources (hydrogen, carbon dioxide and carbon monoxide) for a sulfate reducing bioreactor has been tested on a pilot scale, if not used commercially.

To date the commercial application of the currently available technologies has been limited to a few installations where there are unique waste characteristics or demonstration value. While these technologies have been shown to be effective at waste treatment and metal recovery, costs are relatively high, making the economics marginal. Generally, the most important operating cost will be the carbon and energy source for the bacteria, while the rate of reduction that can be achieved is a key factor in determining plant capacity and therefore capital costs. High reduction rates can generally be achieved using reagent-grade organic energy sources such as ethanol or lactate, but the cost is high for these reagents. Substituting waste organics has been proposed, but locations with an appropriate available source are rare. Readily available lower grade organic wastes such as sewage sludge or agricultural wastes are less biologically available, resulting in lower reduction rates, and these substances can have a variable composition and present material handling problems in bioreactors. Gaseous nutrients, including hydrogen, carbon monoxide and carbon dioxide have been proposed as a lower cost alternative, and tested at large scale, but again reduction rates have been much lower than with organic energy sources.

Gaseous nutrients are generated via the partial oxidation of an available carbon-based fuel, or through steam reforming of light fuels such as methane. These processes involve high temperatures, allowing some heat recovery from the off gas, which can be utilized, for example in maintaining the bioreactor at an optimal operating temperature. When using an organic energy source, additional heating would likely be required to maintain a temperature in the 25-35° C. range, which is an added cost. Typical unit sulfate reduction rates reported with gaseous energy sources have been in the range of 0.1-2 g SO₄ reduced/L of bioreactor volume/day. With organic energy sources, rates of 6-10 g SO₄/L/day have been reported, which translates directly to the bioreactor capacity required to reduce a given quantity of sulfate to sulfide.

Using sulfite as the sulfur source means that the stoichiometric hydrogen requirement for reduction is 75% of that required for sulfate. More importantly, testing with the current process using sulfite as sulfur source has given equivalent reduction rates using gases to those reported for sulfate reduction using organic energy sources. These rates have been obtained with a small-scale laboratory reactor which may not yet have been fully optimized. In addition, the current process has the potential to recapture much more of the energy in the fuel used because the overall process allows the sulfides generated ultimately to be re-combusted after use to regenerate sulfur dioxide. The process also allows for the ultimate combustion of any unused hydrogen and carbon monoxide in the bioreactor off-gas, along with any methane that may have been generated by methanogens that will compete for nutrients in the bioreactor. Due to the nature of bioreactor operation with gaseous nutrients, these can both be serious sources of inefficiency and increased cost, and no process identified in patents or the wider literature has described energy recover from these gases to mitigate the losses.

### Chemical Sulfide Systems:

The basic principles of sulfide precipitation for metal recovery from solutions are well known and chemical sulfide reagents such as sodium sulfide or sodium hydrosulfide have been used for specific applications for many years. One well known example is for nickel and cobalt precipitation in hydrometallurgical processing of nickel concentrates. Use in wastewater treatment or other extractive applications has been limited by the high cost of purchasing, transporting and storing the hazardous sulfide reagents. Precipitates can also be colloidal and difficult to separate. Recent improvements in precipitation and settling have allowed this method to be expanded to those water treatment applications where sufficient high value products such as nickel or copper can be recovered to justify the cost of the reagent.

### Existing Technologies—Standard Non-Sulfide

### Lime Neutralization:

The standard technology for treatment of acidic wastewaters containing heavy metals is neutralization with lime, followed by solid-liquid separation to remove the resulting mixed metal hydroxide/gypsum solid waste. Several versions of this technology are in use, with more recent advances aimed at improving the density and stability of the resulting waste sludge. Metal hydroxide sludges can be very voluminous, and even the solid wastes from high density sludge (HDS) systems can contain a substantial percentage of moisture. Solids resulting from these processes are generally only stable if maintained in an alkaline environment. A drop in pH would result in the re-dissolution of most metals. Also, excessively high pH can result in re-dissolution of certain metals through the formation of hydroxide complexes.

Lime neutralization plants are generally relatively simple and easy to operate, although HDS plants, which are becoming the industry standard, are significantly more complex operations. Operating costs are normally relatively low aside from the reagent costs, which can be substantial when treating a large or highly contaminated waste stream. Lime is a relatively low cost reagent, but is energy intensive to produce and its production is a significant source of carbon dioxide emissions.

When removing metals from solution as hydroxides, each metal has a different pH where its solubility is at a minimum, making it difficult to achieve very low discharge targets for a stream with multiple metals requiring removal. Often the required pH is higher than levels allowed for discharge (e.g. pH 10-11), and a further treatment step is required to lower the solution pH. In general, no values are recovered from lime neutralization processes, and in many cases disposal of the resulting voluminous sludge can be a significant part of the overall treatment cost and can constitute a long term liability for the operator.

Solvent Extraction with Electrowinning:

This technology is used for full scale metal production via solution mining, especially for copper heap leaching. Metal laden pregnant leach solution (often resulting from a sulfuric acid leaching stage) is contacted with an organic solvent in a mix tank and allowed to separate in a settling vessel, transferring dissolved metal to the solvent. The loaded solvent is then contacted with a high strength acid solution to strip the metal out of the solvent, forming a high strength metal solution suitable for direct electrowinning of a final metal product. This technology allows regeneration of acid solutions and production of high value end products with relatively few process steps. The process is well established, being responsible for a significant fraction of world copper production for example.

Process limitations include the high cost of solvent, which is not directly consumed in the process, but suffers regular losses that must be made up. Power for electrowinning is a major process cost, especially where electricity rates are high. The process requires relatively high leachate concentrations (e.g. >2 g/L Cu) to operate effectively, and significant concentrations of contaminants such as iron can be limiting. This results in practical economic limits to the degree of extraction possible at many sites, especially those with slower copper release and/or high iron content, as is often the case for leaching of sulfide ores.

### BRIEF DESCRIPTION OF THE DRAWINGS

FIG. 1 is a simplified design of a bioreactor system.

FIG. 2 is a flow diagram for a sulfide treatment system.

FIG. 3 is a process layout diagram for a treatment system.

### BEST MODE FOR CARRYING OUT THE INVENTION

The basis of the technology is the biologically catalyzed generation of sulfide species (as sulphide ions, hydrosulfide ions or hydrogen sulfide) and alkalinity (as bicarbonate, carbonate or hydroxide ions) through the reduction of divalent, tetravalent or pentavalent sulfur compounds in solution. The sulfide species thus generated can be used for a variety of purposes, which may include the bulk or selective precipitation of metals from metal-contaminated mine drainage; the recovery of metals from hydrometallurgical process streams such as heap leach solutions; or for combustion to generate sulfur dioxide, elemental sulfur, sulfuric acid or any combination of these. It can also be used as a raw material for the production of industrial chemicals such as sodium hydrosulfide (NaHS).

The principal innovations relate to the sulfur and energy balances and where applicable, to the handling of metal sulfide precipitates. In particular, the current invention relies on the generation of partially reduced sulfur compounds through controlled combustion of sulfur-containing fuels. These compounds, or process solutions derived from them, serve as the principal sulfur source for the biological generation of sulfide, allowing important efficiencies to be realized in this core process step.

Biological sulfide generation has been described in the prior art using numerous configurations of sulfur, carbon and energy sources, most commonly using some form of sulfate solution as the sulfur source. The use of solid elemental sulfur, or of sulfur compounds recovered from flue gas desulfurization processes, are both mentioned in the literature. The process described in the claims can utilize any form of partially or fully oxidized sulfur source, including sulfate, but preferably uses one or more less-completely oxidized sulfur species, such as sulfite ions derived from the dissolution of sulfur dioxide gas into solution, or the by-products of using such a solution in a hydrometallurgical process (e.g. sulfite salts, thiosulfate, dithionate or other thiosalts). This may also include the direct injection of a sulfur dioxide-containing gas stream into a bioreactor. The important point of differentiation is that the streams containing these sulfur species are intentionally created as part of an overall process, either as the direct feed to the bioreactor, or as a metallurgical process stream (e.g. for leaching ore) which in turn produces a by-product stream to be fed to the bioreactor.

### Biological Sulfide Generation

The biological stage of the current process consists of one or more anaerobic bioreactors which are fed with the above described sulfur species along with a carbon and energy source, and other nutrients required for bacterial growth. Under these conditions the bacteria reduce the sulfur species to the sulfide form, which is in whole or in part removed from the bioreactor in a gas stream as hydrogen sulfide gas. In the current process the carbon and energy sources for the bacteria are primarily provided by means of a one or more gas streams containing a combination of carbon dioxide, hydrogen, carbon monoxide and nitrogen with little or no oxygen. The gas stream(s) can be produced through the partial oxidation and/or complete oxidation of any carbon-based fuel source alone or in combination with a non-carbon fuel source such as a metal sulfide concentrate under suitable conditions of temperature and pressure, using a controlled amount of oxygen or air. The same gas stream(s) can also provide a portion of the sulfur requirement in the form of sulfur dioxide from the combustion of sulfur in the fuel. The sulfur may be a naturally-occurring component of the fuel, or may be added specifically for this purpose. The undissolved portion of the gas stream(s) (including the nitrogen and depleted amounts of carbon monoxide, hydrogen and carbon dioxide) also acts as a stripping and carrier gas to remove hydrogen sulfide gas from the bioreactor and to transport it to other parts of the process.

The step of partial and/or complete oxidation to produce the bioreactor gas stream(s) is carried out in a suitable high temperature burner(s) with an insulated refractory combustion chamber, with controlled addition of air and/or oxygen to a fluidized, pre-heated feed stream, which may also include a portion of steam injection to enhance the H:C ratio of the resulting bioreactor feed gas. The system may also include a catalytic water-shift reaction stage to convert a portion of the carbon monoxide to hydrogen and carbon dioxide to enhance bioreactor performance, and may include an energy recovery system for reducing the burner exhaust gas to a suitable temperature for bioreactor feed (i.e. reduction from the range of 1000-1500° C. in the burner, to the range 20-50° C. for feed to the bioreactor).

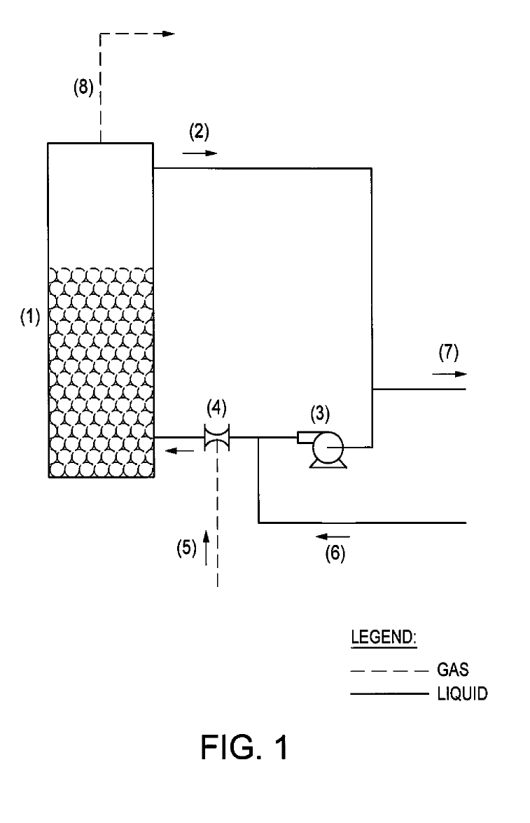

The preferred bioreactor design (FIG. 1) is a packed-bed, up-flow column configuration (1), where the packing is a low-density, high surface area material designed for gas-liquid contacting. Bioreactor overflow (2) solution is recycled by a pump (3) to the bottom of the column where it is re-injected together with the nutrient gas stream (5). The feed inlet line may include in-line mixers or another gas dispersion device such as an educator or jet-pump (4) to ensure efficient dispersion of nutrient gases (5) into the bioreactor solution. On large bioreactors there may be multiple injection points at the base of the column.

In one configuration, the bioreactor feed is a process solution or a waste water stream containing at least one partially-reduced sulfur species such as sulfite (as sulfite salts or as sulfurous acid), dithionate, thiosulfate, etc. This stream may also contain sulfate, although that would not be the primary source of sulfur. In a second, preferred configuration the bioreactor feed solution is a small make-up stream of water containing only dissolved nutrients to support bacterial growth, but containing little or no sulfur (6). In this case the principal source of sulfur would be a direct gas feed containing sulfur dioxide. Nutrients added to the bioreactor will include inorganic mineral salts providing nitrogen, phosphorus and potassium (N, P, K) and other minor or trace nutrients. Any biologically accessible sources of N, P, K and other trace nutrients may be added, but the preferred solution make-up includes the following range of reagent additions: KH₂PO₄ 0.1-2.0 g/L; MgSO₄ 0.1-1.0 g/L; (NH₄)₂SO₄0.1-2.0 g/L or NH₄Cl 0.1-2.0 g/L; CaCl₂ 0.02-0.5 g/L; NaCl 0.1-2.0 g/L; and FeSO₄ 0.01-0.25 g/L. The nutrient feed may also include low levels of an organic substance such as yeast extract or molasses, which is added to support healthy bacterial growth but is not added in concentrations sufficient to act as a significant energy source for the bacteria (e.g. 0.01 to 1.0 g/L of feed solution). One preferred nutrient feed recipe consists of: 0.5 g/L KH₂PO₄, 0.2 g/L MgSO₄, 0.5 g/L (NH₄)₂SO₄, 0.1 g/L CaCl₂, 0.25 g/L NaCl, 0.04 g/L FeSO₄, with 0.1 g/L of molasses. A discharge stream (7) is drawn from the solution recycle stream (2) at a rate matching solution inputs, allowing a constant bioreactor solution volume to be maintained.

### Bioreactor Operation:

When an effective sulfite reducing biomass has been established in the bioreactor, operation consists of maintaining optimal conditions for biological reduction within the bioreactor while supplying a constant supply of fresh sulfite and nutrients. Biomass can ideally be established initially through inoculation with a mixed-population biological sample from an existing active bioreactor, but can also be adapted over time from a broad anaerobic population from a generic source such as an anaerobic sewage sludge digester. Adaptation is achieved by ensuring the presence of sufficient levels of partially or fully oxidized sulfur compounds in the bioreactor solution, along with adequate nutrients for growth as listed above, along with a continuous supply of a carbon and energy source such as carbon dioxide, carbon monoxide and hydrogen. In addition, bioreactor solution pH should be maintained above 7 with the addition of an alkaline reagent if necessary. Competing bacteria such as methanogens, may be preferentially inhibited through the occasional intermittent addition of oxygen to the system. Maintaining significant levels of dissolved sulfide in solution can also be used to help inhibit competing bacteria, although this must limited to avoid inhibiting the desired bacteria. A preferred range for dissolved sulfide in the bioreactor solution is 100-200 mg S²⁻/L.

In addition to ensuring the presence of suitable nutrients, as described above, and a sufficient sulfite concentration (for example above 1 gram SO₃/L of bioreactor volume), it is essential to maintain the bioreactor solution pH in the range of 6.5-9.0. Establishing a suitable initial pH requires that any free sulfurous acid is initially neutralized. After biological activity is well established, the continuous generation of alkalinity allows strongly acidic solutions or gases to be fed to the bioreactor without causing the solution pH to drop below the optimal range. Continuous gas flow is required to maintain the pH, remove reaction products and supply the energy source that drives the reduction. The nutrient feed gas must supply, at a minimum, 3 moles of hydrogen or carbon monoxide for each mole of sulfite fed to the bioreactor. In practise this addition should be significantly higher to account for the low solubility of these gases as well as their uptake by competing bacteria in a mixed population. The ratio of carbon dioxide added in the feed gas stream should be controlled to maintain the bioreactor solution pH in a suitable range.

While the bioreactor can be operated over a wide range of temperatures, biological activity is greatly reduced at lower temperatures. Using a typical mixed mesophilic biomass, the optimal temperature range will be 25-35° C.

Some nutrient competition from methanogens in a mixed biomass is normal, but must be controlled to maintain effective operation. The most effective control method is to ensure a minimum dissolved sulfide concentration of approximately 100 mg/L is maintained in the bioreactor. In a bioreactor with effective continuous gas-stripping of dissolved sulfide, this minimum level is best maintained through an operating pH above 7.5. Excessive levels of dissolved sulfide can also have an inhibiting effect on the sulfite reducing bacteria population, and should be avoided (e.g. greater than 500 mg/L sulfide).

In addition to generating sulfide, the reactions occurring in the bioreactor produce alkalinity mainly in the form of dissolved bicarbonate. Discharge solution containing biologically generated alkalinity is removed from the recycle stream (7) at a rate equivalent to the solution inputs. Depending on the overall process configuration, an aeration vessel may be added to the discharge stream to oxidize any residual sulfide from the bioreactor discharge, and to convert bicarbonate alkalinity to carbonate. In applications where the bioreactor discharge is added into another process stream, this aeration step may not be required.

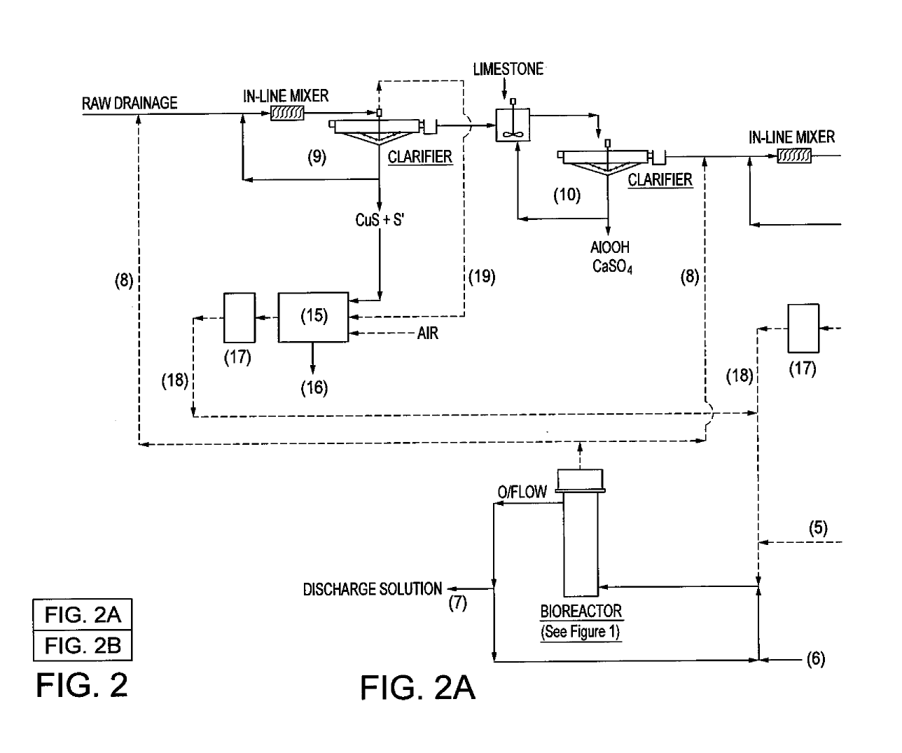

### Preferred Process Embodiment—Sequential Metal Precipitation (FIG. 2)

In the bioreactor biologically catalyzed reactions generate both sulfide and alkalinity. In a preferred configuration, the sulfide is primarily stripped from the bioreactor into an off-gas stream (8) in the form of hydrogen sulfide (commonly 0.1-10% strength). The off-gas stream consists primarily of nitrogen, in combination with residual hydrogen, carbon dioxide and carbon monoxide not taken up by the bacteria. It is also likely to contain a small amount of methane and may carry a hydrogen sulfide content ranging from 0.01 to 15%. This stream can then be used to precipitate metals from waste water or process streams as metal sulfides using a suitable gas-solution contacting device such as an in-line mixer, gas eductor or agitated contacting vessel. The alkalinity generated can also be used to adjust the pH of the waste water or process stream. By controlling the pH of the solution and sulfide addition rate in an appropriate manner, it is possible to produce separate metal precipitates sequentially. The pH may be controlled by addition of alkalinity generated in the bioreactor, or when necessary by the addition of an alkaline reagent such as calcium carbonate, calcium hydroxide, sodium carbonate or sodium hydroxide. The sulfide addition rate may be monitored by measurement of the oxidation-reduction potential of the solution or through the use of an ion specific electrode for sulfide. This technique is now known in the industry, and has been practiced commercially to a limited degree. Also known in the industry are methods for producing an effective solid-liquid separation for removing the metal sulfide precipitates. Most economically important metal sulfides will form colloidal precipitates which can be difficult to settle or filter. By recirculating a portion of the precipitated metal sulfide solids to the gas-solution contacting point, these solids act as seed for the precipitation of the metal sulfide product, resulting in much larger particle sizes and faster settling times. When combined with high-capacity clarifier designs, this allows clarifier footprints and overall plant size to be kept to a minimum.

In water treatment applications, where metal concentrations are low, solids may be recirculated in quantities many times greater than the new solids precipitated from the incoming solution to allow optimal pulp densities to be maintained. A solid concentration of at least 1 g/L by weight at the gas-solution contact point is preferred.

The water treatment and/or metal recovery portion of the process may have one or more metal precipitation stages, and may also include stages without sulfide addition, where pH adjustment and/or carbonate addition is used to remove specific metals or other dissolved solids. At a minimum, each stage consists of a solid-liquid contactor and, if bioreactor off-gas is being added, a gas-liquid contactor such as a stirred tank, eductor, in-line mixer or baffled clarifier feed well, or possibly a combination of these. Each stage also includes a means of solid-liquid separation, such as a clarifier, and slurry pumps for densified sludge recirculation and product discharge.

A preferred process flowsheet for sequential metal removal from acidic waste water such as Acid Rock Drainage (ARD) is shown in FIG. 2. This includes initial selective removal of copper as a sulfide precipitate (9) either by maintaining a low pH (<3.0) or by controlling solution ORP above 0 mV during sulfide gas contacting. The solution pH can then be raised to 4.5-5.5 without further sulfide addition to precipitate aluminum selectively as aluminum hydroxide (10). Alternately raising the pH to >5.5, with aeration will precipitate a combination of ferric iron and aluminum. Addition of sulfide gas in a third stage, while maintaining a pH of approximately 4.0 will produce a selective zinc sulfide precipitate (11). At a pH of 5.5-6.0, nickel and cobalt can be precipitated as a combined sulfide product by again contacting of the solution with sulfide gas (12) with the pH maintained at that level. Iron could be recovered as ferrous sulfide (FeS) by additional sulfide gas contacting with the pH maintained above 6.0, or in the preferred embodiment, as ferric hydroxide (13) by aerating the solution and maintaining the pH at approximately 6.0. The iron could also be recovered as ferrous hydroxide, if required, by increasing the pH above 7.0 without aeration. Finally, from the treated solution, it is possible to precipitate a separate manganese carbonate product by bicarbonate ion addition and pH adjustment to above 8.0 (14). Sulfate levels in solution can be controlled at any stage through the use of a calcium-containing reagent such as lime (CaO) or limestone (CaCO₃) for pH adjustment, resulting in the formation of gypsum (CaSO₄) which has limited solubility. If required, calcium can be removed from the solution through the addition of soluble carbonate or bicarbonate ions at neutral to alkaline pH levels. If present, other hazardous metals such as lead, cadmium, arsenic and chromium can similarly be removed, either selectively or together with other metals, through the appropriate control of solution pH and sulfide addition rates.

High grade metal sulfide concentrates have high energy content, and can be burned to produce metals or metal oxides and sulfur dioxide. This is the basis of commonly used commercial roasting and smelting techniques. In the current invention, sulfide combustion is incorporated into the process. Individual metal sulfide concentrates are dewatered and fed to a combustion device to either: (a) produce metal directly (as in flash smelting) or; (b) generate metal oxides (as in roasting) (15). These combustion processes produce upgraded metal products (16) and also allow energy recovery from the sulfide (17). Also, they produce a sulfur dioxide gas stream (18) which can be fed back to the bioreactor as a sulfur source. In addition to recovering a portion of the energy input to the bioreactor, these combustion devices can be used to combust any unused hydrogen, carbon monoxide or hydrogen sulfide remaining in the process off-gas, further increasing the overall energy efficiency of the technology (19). The re-use of combustion gases in the bioreactor eliminates environmental issues with exhaust gas emissions that are usually associated with sulfide smelting or roasting.

The choice of method for oxidizing the sulfide product will depend on the products being generated and the nature and scale of the application. For example, a burner capable of producing metal directly in a limited oxygen environment would likely be applicable where large amounts of metal product are being generated, such as in a heap leaching application. For smaller-scale operations, such as for water treatment, a simple roasting device using slight excess oxygen to produce a metal oxide product may be more suitable.

One example of a novel advantage of the current invention in the sequential precipitation of metals is in low-pH solutions (pH<3.0) with a significant concentration of dissolved ferric iron (Fe³⁺). In this type of solution, treatment with sulfide will result in the conversion of the iron to the ferrous state (Fe²⁺) and the formation of elemental sulfur. The sulfur precipitates as a solid, while the iron remains in solution. In existing technologies, the copper product grade is diminished by the high sulfur content and expensive sulfide reagent is consumed, or the iron must be removed in a separate pre-treatment, with some loss of other metal values. With the current invention the sulfur is later burned off, recovering energy and returning sulfur to the bioreactor, and the copper product is thus upgraded. With one ARD sample tested (Table 1) the copper sulfide precipitate was heavily diluted with elemental sulfur due to high ferric iron content in the feed solution. Combustion of the sulfur and oxidation of the copper sulfide generated a large amount of potentially recoverable energy and resulted in a product with only 28% of the mass of the original precipitate and a copper grade of nearly 65% and low levels of other contaminants. This is product that would be considered to be a high grade copper concentrate or a suitable feed for an electrowinning stage.

TABLE 1
Effect of combustion on first stage precipitate from ARD
with high copper and high ferric iron levels (Sample B)

| | | Solids | | | | | | | |
|---|---|---|---|---|---|---|---|---|---|
| | Solids | (% of | Al | As | Ca | Cd | Cu | Fe | Zn |
| Product | (g) | feed) | (%) | (%) | (%) | (%) | (%) | (%) | (%) |
| Copper Sulfide Precipitate | 4.01 | 100 | 0.02 | 0.05 | 0.26 | 0.16 | 17.9 | 0.31 | 0.08 |
| Combustion Residue | 1.11 | 27.7 | 0.06 | 0.18 | 0.95 | 0.58 | 64.6 | 1.10 | 0.28 |

The process is not dependant on the oxidation of the metal sulfide precipitates to generate sulfur dioxide. In cases where a metal sulfide is the desired final product (e.g. when a high grade sulfide precipitate is to be combined with an existing metal sulfide concentrate at an operating mine), the bioreactor's sulfur can be derived from another source, such as a high sulfur fuel chosen with sufficient sulfur content to meet the process sulfide requirement in addition to providing the carbon and energy source for the bioreactor.

In applications where metal product value is the principal objective, additional steps can be added to clean, separate and/or upgrade the resulting metal products. Following separation of the sulfide precipitate, this can include washing with water or dilute acid solutions to remove impurities. During oxidation, temperatures can be controlled to separate and collect any trace volatile metal impurities that may be present, such as mercury or cadmium. Finally, the resulting metal oxide product can be re-leached with an acid or alkaline lixiviant to form a clean concentrated dissolved metal solution suitable for producing a final product, either by electro-winning the pure metal or by chemical precipitation of a desired chemical product (e.g. copper sulfate, cobalt hydroxide, etc.).

### EXAMPLES

### Example 1

### Bioreactor Operations

The unexpected result of using tetravalent sulfur i.e. sulfite as a bioreactor sulfur source rather than sulfate has been demonstrated in laboratory-based continuous testing. The unit sulfite reduction rates obtained have been significantly higher than sulfate reduction rates that have been obtained under similar conditions. Typical reduction rates for sulfate using inorganic gaseous nutrients are in the range of 1.0-1.2 grams SO₄ per litre of bioreactor volume per day. The selected example data shows the results from high-level operation over a 10 day period. Reduction rates were determined to be as much as 5 times higher with sulfite as compared with sulfate and even higher when the equivalent weight of sulfate was considered that would result in the same amount of sulfide. This higher-than-expected reduction rate is important in making the use of low-cost gaseous nutrients an effective process option, which has important economic benefits for the use of the process.

TABLE 2
Laboratory Bioreactor Operating Data

| | | | | | | | | SO₄ |
|---|---|---|---|---|---|---|---|---|
| | Feed | | | Bioreactor | | SO₃ Reduction Rate | | Equiv. |
| | Rate | | SO₃ | | SO₃ | Daily | 3-Day Ave. | 3-Day Ave. |
| Date | (mL/day) | pH | (g/L) | pH | (g/L) | (g/L/day) | (g/L/day) | (g/L/day) |
| 16-Apr | 240 | 2.93 | 32.83 | 8.30 | 6.67 | 6.56 | 5.26 | 6.32 |
| 18-Apr | 226 | 2.93 | 33.08 | 8.50 | 4.59 | 5.82 | 5.73 | 6.87 |
| 20-Apr | 250 | 2.93 | 33.08 | 8.40 | 4.28 | 5.42 | 5.93 | 7.12 |
| 23-Apr | 258 | 2.93 | 33.08 | 8.40 | 3.17 | 5.98 | 5.74 | 6.88 |
| 25-Apr | 260 | 3.00 | 33.08 | 8.40 | 3.42 | 5.50 | 5.63 | 6.76 |

A unique part of the invention is the intentional generation of a process stream containing partially reduced sulfur products, such as a combustion off-gas stream containing sulfur dioxide. The current invention allows these streams to be produced under controlled conditions that, for example, limit the amount of excess oxygen present. This reduces the potential for further oxidation of sulfite to sulfate, which would limit its effectiveness for use in the bioreactor. Also, a stream that contains a significant amount of excess oxygen would not be a suitable feed for direct use in the bioreactor. As an illustration of the negative effect of high oxygen content in a gas stream fed to the bioreactor, a test was conducted in which the nitrogen component of the gas stream fed to a laboratory bioreactor was replaced by air for an 18 hour period. This resulted in an overall gas mixture that contained 5.9% oxygen without changing the addition rates of carbon dioxide and hydrogen. The results of bioreactor operation over a ten day period that included this 18 hour test are shown in Table 3.

The reduction in bioreactor performance is apparent during the test period with a drop in pH due to reduced alkalinity generation. Even after the conclusion of the test the bioreactor performance continues to be negatively affected for several days, as indicated by the significantly lower sulfite reduction rates. Nearly a week is required to fully recover from this 18 hour test.

TABLE 3
Effects of oxygen in feed gas to bioreactor
Test Period: May 3-4: 18 Hours
Gas Flows: CO₂/H₂ mix: 460 mL/min.
Air: 180 mL/min.
O₂ content in gas: 5.9%

| | | | | | | | | SO₄ |
|---|---|---|---|---|---|---|---|---|
| | Feed | | | Bioreactor | | SO₃ Reduction Rate | | Equiv. |
| | Rate | | SO₃ | | SO₃ | Daily | Week Ave. | 3-Day Ave. |
| Date | (mL/day) | pH | (g/L) | pH | (g/L) | (g/L/day) | (g/L/day) | (g/L/day) |
| 2-May | 252 | 3.00 | 31.42 | 8.30 | 7.25 | 5.29 | 4.26 | 5.11 |
| 4-May | 266 | 3.00 | 28.58 | 7.90 | 7.67 | 4.29 | 4.69 | 5.63 |
| 7-May | 228 | 3.00 | 28.58 | 8.10 | 13.58 | 1.31 | 3.63 | 4.36 |
| 9-May | 240 | 2.80 | 28.58 | 8.10 | 13.67 | 3.66 | 3.09 | 3.71 |
| 11-May | 235 | 2.80 | 37.75 | 8.10 | 13.33 | 5.35 | 3.44 | 4.13 |

### Example 2

### Process Applications

The invention has many possible applications in the metal and chemical industries and in environmental applications. Without limiting the scope of use claimed for the invention, the following examples describe the principal preferred process embodiments. Additional applications are likely where the availability of a low-cost sulfide reagent provides an economic benefit.

### Mine Drainage—Full Treatment:

This involves the use of biogenic sulfide and alkalinity for the complete treatment of mine drainage to discharge quality. This may include sequential precipitation of multiple metal products as sulfides, hydroxides and/or carbonates, and may include some addition of supplemental alkalinity, such as limestone, lime, caustic soda or soda ash, for pH adjustment and possibly for gypsum precipitation to reduce total dissolved solids (TDS) by removing sulfate. It may also include aeration of certain stages to adjust the solution ORP potential. Most common metal contaminants can be removed effectively through an appropriate combination of pH adjustment and sulfide addition or aeration.

In laboratory testing, two separate mine drainage types were tested, one representing a high-flow, dilute stream with limited contaminants (Sample A) and the other a highly acidic stream with high metal loading. Tables 4-4C show the characteristics of these samples and the results of process configurations for selective metal precipitation. Table 4 shows the initial composition of the two samples tested and Table 4A shows the results from selective precipitation of copper and zinc from Sample A. Sample B was tested in two different configurations, one meant to recover separate copper and zinc products while treating the water to discharge quality (Table 4B), and the other to show the ability to produce separate products even for lesser contaminants with recoverable value, including cobalt, nickel and manganese (Table 4C).

TABLE 4
Analysis of mine drainage samples used for metal precipitation testing.

| | Flow | | Cu | Zn | Co | Ni | Cd | Al | Fe | Mn |
|---|---|---|---|---|---|---|---|---|---|---|
| Sample | (m³/day) | pH | (mg/L) | (mg/L) | (mg/L) | (mg/L) | (mg/L) | (mg/L) | (mg/L) | (mg/L) |
| Sample A - High Flow | 12,000 | 4.3 | 14.3 | 17.8 | <0.1 | <0.1 | 0.09 | 13.3 | 0.4 | 3.65 |
| Sample B - High Strength | 2,500 | 2.7 | 67 | 181 | 4.65 | 9.68 | 1.37 | 950 | 872 | 184 |

TABLE 4A
Sample A - Three product configuration, 1 litre test (metal values
indicate % removal from solution, bd—bioreactor discharge solution).

| | | Reagents | Addition | Cu | Zn | Cd | Al |
|---|---|---|---|---|---|---|---|
| Stage | pH | Added | (g/l) | (%) | (%) | (%) | (%) |
| Feed | 4.3 | — | — | 0 | 0 | 0 | 0 |
| Copper | 3.7 | H₂S | — | 98 | 0 | 56 | 0 |
| Recovery | | | | | | | |
| Zinc Recovery | 4 | H₂S/bd | bd: 3.7 | 100 | 98 | 100 | 5 |
| pH Adjustment | 6.2 | bd | 70.4 | 100 | 100 | 100 | 98 |

TABLE 4B
Sample B - Four stage, three product configuration, 2 litre
test (metal values indicate % removal from solution).

| | | Reagent | Addition | Cu | Zn | Co | Ni | Cd | Al | Fe | Mn |
|---|---|---|---|---|---|---|---|---|---|---|---|
| Stage | pH | Added | (g/l) | (%) | (%) | (%) | (%) | (%) | (%) | (%) | (%) |
| Feed | 2.6 | — | — | 0 | 0 | 0 | 0 | 0 | 0 | 0 | 0 |
| Copper Recovery | 2.4 | H₂S | — | 100 | 0 | 0 | 0 | 100 | 0 | 0 | 0 |
| Al/CaSO₄ | 5 | CaCO₃ | 5 | 100 | 4 | 0 | 0 | 100 | 99 | 0 | 1 |
| Removal | | | | | | | | | | | |
| Zinc Recovery | 3 | H₂S | — | 100 | 97 | 0 | 0 | 100 | 98 | 0 | 1 |
| Final Treatment | 7.8 | CaO | 4.5 | 100 | 100 | 98 | 99 | 100 | 100 | 100 | 42 |

TABLE 4C
Sample B -Multi stage configuration for minor metal recovery, 2 litre test (metal
values indicate % removal from solution, bd—bioreactor discharge solution).

| | | Reagent | Addition | Cu | Zn | Co | Ni | Cd | Al | Fe | Mn |
|---|---|---|---|---|---|---|---|---|---|---|---|
| Stage | pH | Added | (g/l) | (%) | (%) | (%) | (%) | (%) | (%) | (%) | (%) |
| Feed | 2.7 | — | — | 0 | 0 | 0 | 0 | 0 | 0 | 0 | 0 |
| Copper Recovery | 2.3 | H₂S | — | 100 | 0 | 0 | 0 | 100 | 0 | 0 | 0 |
| Al/CaSO₄ | 5.1 | CaCO₃ | 5.3 | 100 | 9 | 0 | 0 | 100 | 99 | 1 | 0 |
| Removal | | | | | | | | | | | |
| Zinc Recovery | 3 | H₂S | — | 100 | 95 | 0 | 0 | 100 | 99 | 1 | 0 |
| Co—Ni Recovery | 5.1 | H₂S/CaCO₃ | 0.21 | 100 | 100 | 53 | 71 | 100 | 99 | 1 | 0 |
| Iron Removal | 7.7 | CaO | 1.9 | 100 | 100 | 99 | 99 | 100 | 100 | 100 | 81 |
| Mn Recovery | 8.3 | bd | 57 | 100 | 100 | 98 | 99 | 100 | 100 | 100 | 95 |

### Mine Drainage—Partial Treatment:

In many cases, mine drainage streams are currently being treated using some form of lime neutralization technology. Often these streams include one or more valuable metals in significant concentrations that can be recovered through the addition of a sulfide precipitation stage to the existing treatment plant, resulting in metal recovery and a decrease in lime consumption and solid waste generation. In other cases certain toxic heavy metals are present at levels that are difficult to remove with lime treatment alone, and sulfide precipitation can be added to remove these metals to meet discharge requirements. An example is cadmium, which cannot always be removed to required levels with the use of lime.

### Heap Leaching—Full Recovery:

Current technology for base metal recovery from heap leaching operations is solvent extraction and electrowinning (commonly used for copper recovery). The present technology can be used as an alternative method for direct metal recovery from the leach solution as a sulfide, followed by combustion of the sulfide precipitate to produce either a metal oxide product (suitable for re-dissolution in acid and electrowinning of metal), or a raw metal product (suitable for electrorefining). This would particularly suit copper heap leaching, where sulfide precipitation will regenerate the acid leach solution while leaving most potential impurities in solution.

Heap Leaching—Bleed Stream and/or Life Extension:

Conventional solvent extraction technology requires certain minimum metal concentrations in leach solutions to operate effectively. For copper, this is typically reported to be approximately 2 grams of copper per liter of leach solution. Sulfide precipitation can be operated effectively at levels below 100 milligrams of copper per liter of leach solution. In addition, when impurities are not removed from the regenerated leach solutions, they build up and may need to be managed by removal and treatment of a bleed stream. The present technology can be added to existing heap leach operations for the treatment of bleed streams, and could also be used to replace solvent extraction later in the life of the heap, when metal concentrations have dropped below economic levels for solvent extraction, thus extending the project life and increasing the overall recovery.

### Conventional Hydrometallurgical Leaching Processes:

Chemical sulfide precipitation is currently used for selective metal recovery in some leaching processes, especially for nickel and cobalt recovery. The present technology can be utilized as an alternative source for sulfide in these processes, and by improving the availability of low-cost sulfide, could also be expected to expand the use of sulfide precipitation, both for primary metal recovery and for treatment of waste and bleed streams.

Metal Recovery from Industrial Process Streams:

Many industrial processes generate metal contaminated waste streams that could benefit from treatment with sulfide precipitation to recover valuable metals. Examples include electroplating and metal finishing wastes, electronic manufacturing and circuit board etching wastes, etc. Industrial processes may also include treatment of solid wastes, such as electronic scrap, for removal and recovery of heavy metals, where sulfide precipitation could be used to recover specific metals from leach streams.

Leaching with Reduced Sulfur Species:

In specific mineral extraction processes one or more leach solutions may be used which contain one or more partially reduced sulfur species such as sulfur dioxide/sulfites (for example as sulfurous acid), or alkaline thiosulfate solutions. In these applications the current process may have the dual purpose of recovering leached metals from solution and regenerating leaching agents. For example, spent leach solutions can be fed to the bioreactor to generate hydrogen sulfide gas, which can then be used as raw material in generating fresh sulfur dioxide or thiosulfate.

### Petroleum Coke Utilization:

The coke residue from upgrading heavy oils such as bitumen from oil sands has high fuel value due to the carbon content, but also has high sulfur and ash content which limits its beneficial use. Partial oxidation combustion of petroleum coke could, however, provide an effective feed gas to a bioreactor allowing energy recovery and conversion of the sulfur to sulfide, which could be used as an industrial chemical, or converted to elemental sulfur or other sulfur products. In addition, the ash from petroleum coke is often high in valuable metals such as nickel and vanadium. With a suitable leaching stage, these metals could also be recovered as separate products.

### Example 3

### Balance of Inputs from Mixed Gas Streams in a Typical Bioreactor Operation in Winter Conditions

In an application of the invention for metal precipitation from wastewater at a minesite the required inputs to the bioreactor will include heat to maintain an optimal solution temperature, a sufficient supply of reduced sulfur species to meet the plant H₂S requirements, an energy source sufficient to complete the reduction to H₂S, a carbon source and trace nutrients for biomass growth and sufficient carbon dioxide to maintain the bioreactor pH at the desired level.

Various researchers have suggested a range of optimal temperatures for biologically catalyzed sulfide generation (Baskaran 2005), with variations likely resulting from different dominant strains of microorganisms, substrates and bioreactor designs. Despite these variations, reported optimal operating conditions normally lie within the range of 25 to 35° C. Available research (Sawicka 2012) indicates that reduction rates can be expected to show a near linear decrease until activity ceases entirely near 0° C. Thus under winter conditions at the example minesite, bioreactor heating will be required where the ambient air and water temperatures will be well below the optimum levels.

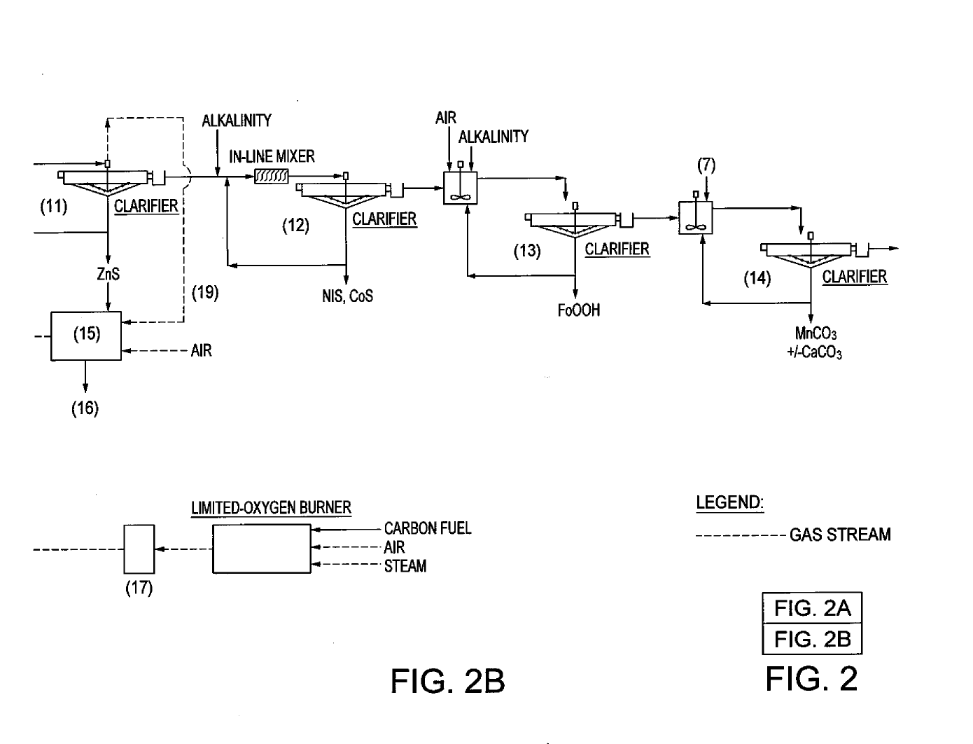

For this example application (FIG. 3) the process requirement is to generate in excess of 300 kg/day of H₂S for the precipitation of copper sulfide (101) from a wastewater stream (102). The copper is precipitated as per the reaction:

CuSO₄(aq)+H₂S(g)→CuS₂S+H₂(SO₄(aq)

After precipitation, the CuS is dewatered (103) and burned without excess air (104) as per Example 4 to produce a CuO product (105) and a gas stream containing N₂, SO₂ and heat (106), which is fed to the bioreactor together with a separate gas stream to provide a carbon and energy source. The SO₂, which is highly soluble, dissolves in the bioreactor to form hydrogen sulfite:

SO₂(g)+H₂O(l)→H₂SO₃(aq)

The N₂ in the gas stream passes through the bioreactor and acts as a carrier gas to remove the H₂S that is generated by biological sulfite reduction (107). This gas stream is carried back to the CuS precipitation stage (101).

The carbon and energy for the bioreactor is provided by the partial oxidation of methane (108) with steam (109) and limited air (110) in a partial oxidation burner (111) to produce a gas stream containing H₂ and CO₂ along with N₂, H₂O and some excess heat (112) as in Example 6. In the bioreactor (113) the sulfite is reduced according to the generalized formula:

3H₂(aq)+SO₃²⁻(aq)→H₂S(aq)+H₂O(l)+2OH⁻(aq)

The CO₂ present in the gas stream dissolves in solution to form bicarbonate ions:

CO₂(g)+H₂O(l)→H⁺(aq)+HCO₃⁻.(aq)

Bicarbonate can be taken up by sulphite reducing bacteria to form new cell mass during growth. It also serves to regulate the solution pH by neutralizing hydroxide ions:

OH⁻(aq)+CO₂(g)→HCO₃⁻.(aq)

The bioreactor is located outdoors, with an ambient winter temperature of −4° C. and plant process water is available to feed nutrients to the bioreactor at 10° C. The bioreactor is insulated to minimize heat loss. The bioreactor is maintained at an optimal temperature of 30° C., which allows a sulfite reduction rate of 6.0 g SO₃ red./L/day to be maintained. To provide the required amount of H₂S a bioreactor of 125 m3 is required (4 meter diameter and 10.5 meters high with a 0.5 meter gas head space). For an insulated tank in this environment, the total heat loss is 10.0 kW (Ogden, 2012). Nutrient feed solution (114) at 10° C. is constantly fed to the bioreactor at a rate of 1.3 m³/hr, which requires an additional 29.3 kW to heat to 30° C.

The bioreactor takes up H₂ at an efficiency of 60%, which results in a total H₂ requirement of 94.2 kg/day. This is provided by the partial oxidation of 251 kg/day of CH₄ as in Example 6.

From Example 4 below, oxidation of CuS will provide 2.40 kWh/kg H₂S of available heating energy, while partial oxidation of CH₄ provides an additional 1.04 kWh/kg H₂S. For the operating conditions in this example a total of 943 kWh/day of heating is required to produce 320 kg/day of H₂S. This can be met by the 1100 kWh of available heat energy in the two bioreactor feed gas streams. Excess heat can be utilized for plant or process heating (117) by use of heat exchangers (116) on the gas streams and the bioreactor discharge stream (115).

During warmer ambient conditions, when much more excess heat is available, a waste heat boiler (118) can be utilized to generate steam (119) for plant use or power generation.

### Example 4

### Production of Sulfur Dioxide Containing Flue Gas from Burning a Metal Sulfide with a Stoichiometric Amount of Air

The production of sulfur dioxide containing combustion flue gas that will form part of useable biological nutrients can be accomplished by burning a metal sulfide. This will also generate heat that can be used to maintain optimal bioreactor temperatures. As an example, burning copper sulfide stoichiometrically with air will proceed according to the following reaction:

CuS(s)+O₂(g)+3.76N₂(g)→CuO(s)+SO₂(g)+3.76N₂(g)

The SO₂ containing flue gas will form part of useable biological nutrients. The heat of reaction ΔHᵣₓₙ, is calculated as the sum of the enthalpy of formation, ΔH_{f}, for each product compound minus the sum of the ΔH_{f} for each reactant compound. The ΔH_{f} for each compound is provided in Table 5 (Perry et al, 1999 pp 2-187 to 2-195).

TABLE 5
Enthalpy of Formation

| Compound | CuS | O2 | N₂ | CuO | SO₂ |
|---|---|---|---|---|---|
| ΔH_{f} (KJ/mol) | −53.1 | 0.0 | 0.0 | −157.3 | −296.81 |

ΔHᵣₓₙ=[(−157.3)+(−296.81)+(3.76(0.0))]−[(−53.1)+(0.0)+(3.76(0.0))]=−401.01 KJ/mol

The ΔHᵣₓₙ provides energy to elevate the temperature of the final products. Accurate calculation of the high temperature heat content for the final products requires heat capacity data which can be mathematically integrated between ambient temperature and final temperature. The heat content in each of compound in the final product is summed to calculate the total heat content of the final product:

Heat in Final Products=Σnᵢ∫CₚDT
Where: nᵢ=number of moles of each final product
Cₚ=heat capacity of each final product
   Cₚ data for each compound in the final products are provided in Table 6 (Perry et al 1999 pp 2-161 to 2-168).

TABLE 6
Heat Capacity of Compounds

| | Cp (calorie / degree Kelvin mole); T = K, |
|---|---|
| Compound | 0° C. = 273.1 K; 4.18 Joules = 1 calorie |
| CuO(s) | 10.87 + 0.003576T − 150600 / T² |
| SO₂(g) | 7.70 + 0.00530T − 0.00000083 T² |
| N₂(g) | 6.50 + 0.00100 T |

The high temperature heat content for each product compound is equivalent to the mathematically integrated Cₚ, for which equations are provided in Table 7.

TABLE 7
High Temperature Heat Content of Product Compounds

| | ∫ Cp DT (calorie / mole); T = K, |
|---|---|
| Compound | 0° C. = 273.1 K; 4.18 Joules = 1 calorie |
| CuO(s) | [10.87T_{final} + (0.003576/2)T²_{final} + (150600 / T_{final})] − |
| | [10.87Tᵢₙᵢₜᵢₐₗ + (0.003576/2)T²ᵢₙᵢₜᵢₐₗ + (150600 / Tᵢₙᵢₜᵢₐₗ)] |
| SO₂(g) | [7.70Tₜᵢₙₐₗ + (0.00530/2)T²_{final} − (0.00000083 / 3)T³_{final}] − |
| | [7.70Tᵢₙᵢₜᵢₐₗ + (0.00530/2)T₂ᵢₙᵢₜᵢₐₗ − (0.00000083 / 3)T³ᵢₙᵢₜᵢₐₗ |
| N₂(g) | [6.50T_{final} + (0.00100/2) T²_{final}] − |
| | [6.50Tᵢₙᵢₜᵢₐₗ + (0.00100/2) T²ᵢₙᵢₜᵢₐₗ] |

The final temperature of the products, T_{final}, can by determined by balancing the ΔHᵣₙₓ with the total high temperature heat content of the final products.

ΔHᵣₙₓ=Σnᵢ∫CₚDT

Using an initial ambient temperature, Tᵢₙᵢₜᵢₐₗ, of 25° C., T_{final} can be solved by iteration and determined to be 1737° C.

The heat from the exhaust gases (SO₂ and N₂) can be heat exchanged with the bioreactor to maintain optimal operating temperature. The available heat in this exhaust gas can be calculated by adding the individual high temperature heat content of each exhaust gas components (SO₂ and N₂) using the equations in Table 7. By inputting an exhaust gas temperature of 1737° C. and a bioreactor process temperature of 30° C., the heat available from the exhaust gas to maintaining optimal bioreactor temperature is calculated to be 294.78 KJ per mole of SO₂ output.

Given that 1 mole of SO₂ input into the bioreactor will provide 1 mole of H₂S output and the molecular weight of H₂S being 34.0809 grams per mole, the total available heat from the exhaust gases obtained from the stoichiometric burning of CuS with air is calculated to provide 2.40 KWh of Heat per kilogram of H₂S output.

The CuO product is a suitable feed for an independent closed loop electrowinning circuit to produce Cu metal. CuO is readily dissolved in acid (H⁺), whereby the acid is regenerated in the electrowinning circuit. The aqueous reaction to make electrolyte for electrowinning is as follows:

CuO+2H⁺→Cu²⁺+H₂O

Cu metal is electroplated onto a cathode of an electrowinning cell, whereas H⁺ is regenerated on the anode of the electrowinning cell. The Cu metal is harvested for its value and the H⁺ is reused to make up more electrowinning electrolyte by dissolving more CuO. The cathode and anode half cell reactions for electrowinning copper are as follows:

Cathode Reaction: Cu²⁺+2e⁻→Cu (metal)

Anode Reaction: H₂O→½O₂+2H⁺+2e⁻

The net electrowinning reaction will be:

Cu²⁺+H₂O→Cu (metal)+½O₂+2H⁺

A suitable acid for Cu electrowinning is H₂SO₄. In this case, the overall reaction for making the electrolyte for electrowinning is:

CuO+H₂SO₄→CuSO₄⁺H₂O

The overall reaction for electrowinning is:

CuSO₄⁺H₂O→Cu (metal)+½O₂+H₂SO₄

### Example 5

### Production of Sulfur Dioxide Containing Flue Gas from Burning a Metal Sulfide with 10% Excess Air

The burning of copper sulfide with 10% excess air will proceed according to the following reaction:

CuS(s)+1.1O₂(g)+4.14N₂(g)→CuO(s)+SO₂(g)+4.14N₂(g)+0.1O₂(g)

Using the molar ratio of the products of reaction, the exhaust gas is calculated to contain 19.1% SO₂, 79% N₂ and 1.9% O₂.

Using the method described in Example 4, the temperature of the reaction products is calculated to be 1664° C.

Using the method described in Example 4, the heat available from the exhaust gas to maintaining optimal bioreactor temperature is calculated to be 2.51 KWh of Heat per kilogram of H₂S output.

### Example 6

### Production of Carbon Dioxide and Hydrogen Containing Flue Gas by Burning Methane with Water and Stoichiometric Air

The production of carbon dioxide and hydrogen containing combustion flue gas that will form part of useable biological nutrients can be accomplished by burning methane with water. This will also generate heat that can be used to maintain optimal bioreactor temperatures. As an example, burning methane and water stoichiometrically with air will proceed according to the following reaction:

CH₄(g)+H₂O+0.5O₂(g)+1.88N₂(g)→CO₂(g)+3H₂(g)+1.88N₂(g)

Based on the molar ratio of the final products, the exhaust gas will contain 17.0% CO₂, 51.0% H₂ and 32.0% N₂. With addition thermodynamic data available from Perry et al and using the same method of calculation described in Example 4, the ΔHᵣₓₙ is calculated to be −77.2 KJ per mole CO₂ (or 3 mole H₂) produced.

Using the method described in Example 4, the temperature of the reaction products is calculated to be 435° C.

Combining this exhaust gas with another exhaust gas containing sulfur dioxide, such as the one described in Example 4, will produce a composite gas stream that contain useable quantities biological nutrients or energy sources for the production of H₂S.

Given the estimated requirement of 147.0 moles of H₂ required per kilogram H₂S produced (described in Example 3), burning sufficient fuel to produce the required H₂ will also provide 1.04 KWh of heat in it's exhaust gas to per kg H₂S bioreactor output.

If the CO₂ from this reaction is used to maintain the pH of the bioreactor, 2 moles of CO₂ would be required for every mole of H₂S produced in the bioreactor. If the amount of fuel is balanced with the hydrogen demand, this exhaust gas will provide 1.67 of the required 2 moles of CO₂ required for pH control. Adjusting the fuel to air ratio can better balance the required H₂ and CO₂ required for the biological generation of H₂S.

### Example 7

### Production of Carbon Monoxide, Carbon Dioxide and Hydrogen Containing Flue Gas by Burning Methane with Sub-Stoichiometric Air

The production of carbon monoxide, carbon dioxide and hydrogen containing combustion flue gas that will form part of useable biological nutrients can be accomplished by burning methane with sub-stoichiometric air. This will also generate heat that can be used to maintain optimal bioreactor temperatures. As an example, burning methane sub-stoichiometrically with air will proceed according to the following reaction:

CH₄(g)+0.75O₂(g)+2.82N₂(g)→0.5CO(g)+0.5CO₂(g)+2H₂(g)+2.82N₂(g)

Based on the molar ratio of the final products, the exhaust gas will contain 8.6% CO, 8.6% 34.4% H₂ and 48.4% N₂. With addition thermodynamic data available from Perry et al and using the same method of calculation described in Example 4, the ΔHᵣₓₙ is calculated to be −177.5 KJ per mole CH₄ burned.

Using the method described in Example 4, the temperature of the reaction products is calculated to be 974° C.

Combining this exhaust gas with another exhaust gas containing sulfur dioxide, such as the one described in Example 4, will produce a composite gas stream that contain useable quantities biological nutrients or energy sources for the production of H₂S.

Given the estimated requirement of 147.0 moles of H₂ required per kilogram H₂S produced (described in Example 3), burning sufficient fuel to produce the required H₂ will also provide 3.3 KWh of heat in it's exhaust gas to per kg H₂S bioreactor output.

Assuming that the CO is converted to CO₂ in the bioreactor and that this CO₂ will be used to maintain the pH of the bioreactor, 2 moles of CO₂ would be required for every mole of H₂S produced in the bioreactor. If the amount of fuel is balanced with the hydrogen demand, this exhaust gas will provide 2.5 moles CO₂, whereas only 2 moles of CO₂ is required for pH control. Adjusting the fuel to air ratio can better balance the required H₂ and CO₂ required for the biological generation of H₂S.

### Example 8

### Production of Sulfur Dioxide Containing Flue Gas from Burning Zinc Sulfide with a Stoichiometric Amount of Air

The production of sulfur dioxide containing combustion flue gas that will form part of useable biological nutrients can be accomplished by burning a zinc sulfide. This will also generate heat that can be used to maintain optimal bioreactor temperatures. As an example, burning zinc sulfide stoichiometrically with air will proceed according to the following reaction:

ZnS(s)+O₂(g)+3.76N₂(g)→ZnO(s)+SO₂(g)+3.76N₂(g)

With additional thermodynamic data available from Perry et al (pp 2-187 to 2-195) and using the same method of calculation described in Example 4, the ΔHᵣₓₙ is calculated to be −441.29 KJ per mole SO₂ produced.

Using the method described in Example 4, the temperature of the reaction products is calculated to be 1947° C.

Using the method described in Example 4, the total available heat from the exhaust gases obtained from the stoichiometric burning of ZnS with air is calculated to provide 2.74 KWh of Heat per kilogram of H₂S output.

The ZnO product is a suitable feed for an independent closed loop electrowinning circuit to produce Zn metal. ZnO is readily dissolved in acid (H⁺), whereby the acid is regenerated in the electrowinning circuit. The aqueous reaction to make electrolyte for electrowinning is as follows:

ZnO+2H⁺→Zn²⁺+H₂O

Zn metal is electroplated onto a cathode of an electrowinning cell, whereas H⁺ is regenerated on the anode of the electrowinning cell. The Zn metal is harvested for its value and the H⁺ is reused to make up more electrowinning electrolyte by dissolving more ZnO. The cathode and anode half cell reactions for electrowinning zinc are as follows:

Cathode Reaction: Zn²⁺+2e⁻→Zn (metal)

Anode Reaction: H₂O→½O₂+2H⁺+2e⁻

The net electrowinning reaction will be:

Zn²⁺+H₂O→Zn (metal)+½O₂+2H⁺

A suitable acid for Zn electrowinning is H₂SO₄. In this case, the overall reaction for making the electrolyte for electrowinning is:

ZiO+H₂SO₄→Z₂SO₄→ZnSO₄+H₂O

The overall reaction for electrowinning is:

ZnSO₄+H₂O→Zn (metal)+½O₂+H₂SO₄

### Example 9

### Production of Carbon Dioxide and Sulfur Dioxide Containing Flue Gas by Burning Petroleum Coke with Stoichiometric Air

The production of carbon dioxide and sulfur dioxide containing combustion flue gas that will form part of useable biological nutrients can be accomplished by burning petroleum coke. This will also generate heat that can be used to maintain optimal bioreactor temperatures.

The composition of petroleum coke varies with the crude from which it is made. The range of composition is provided in Table 8 (Singer, 1991 pp 2-22).

TABLE 8
Composition of Petroleum Coke

| | Minimum % | Maximum % |
|---|---|---|
| Composition | by Weight | by Weight |
| Moisture | 3 | 12 |
| Volatile Matter | 10 | 20 |
| Fixed Carbon | 71 | 88 |
| Ash | 0.2 | 3.0 |
| Sulfur | 2.9 | 5.4 |

For the purpose of this example calculation, only fixed carbon (88%), sulfur (5.4%) and moisture (6.6%) are considered.

For the basis of 1 kilogram of petroleum coke and taking into account the molecular weights of each component used in this calculation, the stoichiometric burning of petroleum coke will occur according to the following reaction:

73.3C+1.7S+75.0O₂+282.0N₂+3.7H₂O→73.3CO₂+1.7SO₂+282.0N₂+3.7H₂O

Based on the molar ratio of the final products, the exhaust gas will contain 20.3% CO₂, 0.5% SO₂, 78.2% N₂ and 1.0% H₂O. With addition thermodynamic data available from Perry et al and using the same method of calculation described in Example 4, the ΔHᵣₓₙ is calculated to be −29,331.5 KJ per Kg petroleum coke burned.

Using the method described in Example 4, the temperature of the reaction products is calculated to be 2158° C.

Combining this exhaust gas with another exhaust gas containing sulfur dioxide, such as the one described in Example 4, will produce a composite gas stream that contain useable quantities biological nutrients or energy sources for the production of H₂S. Extra SO₂ produced from the burning of petroleum coke will be provided to the composite exhaust stream for nutrient feed into the bioreactor for greater production of H₂S. Since this reaction does not produce H₂, this must be added from another source into the composite gas stream.

If the CO₂ from this reaction is used to maintain the pH of the bioreactor, 2 moles of CO₂ would be required for every mole of H₂S produced in the bioreactor. With this fuel requirement and using the calculation method described in Example 4, the heat available from the exhaust gas to maintaining optimal bioreactor temperature is calculated to be 6.43 KWh of Heat per kilogram of H₂S output.

### Example 10

### Burning of Petroleum Coke with Sub-Stoichiometric Air

The burning of petroleum coke (with the same composition as described in Example 9) with sub-stoichiometric air will proceed according to the following reaction

73.3C+1.7S+38.3O₂+144.2N₂+3.7H₂O→66.25CO+7.0CO₂+1.7H₂S+144.2N₂+1.98H₂

Based on the molar ratio of the final products, the exhaust gas will contain 30.0% CO, 3.2% CO₂, 0.8% H₂S, 65.2% N₂ and 0.9% H₂. With addition thermodynamic data available from Perry et al and using the same method of calculation described in Example 4, the ΔHᵣₓₙ is calculated to be −9276.4 KJ per Kg petroleum coke burned.

Using the method described in Example 4, the temperature of the reaction products is calculated to be 1238° C.

Combining this exhaust gas with another exhaust gas containing sulfur dioxide, such as the one described in Example 4, will produce a composite gas stream that contain useable quantities biological nutrients or energy sources for the production of H₂S.

Note that the burning of sulfur containing petroleum coke does not form SO₂ when insufficient air is used in combustion. In this particular condition, the exhaust gas is high CO in comparison to CO₂. This exhaust gas also has a small amount of H₂ and H₂S. The majority of the nutrient in this exhaust gas will be from CO, which the bioreactor will convert to H₂ with by the following water shift reaction.

CO+H₂O→CO₂+H₂

Given the estimated requirement of 147.0 moles of H₂ required per kilogram H₂S produced (described in Example 3), burning sufficient fuel and subsequent water shift to produce the required H₂ will also provide 3.59 KWh of heat in it's exhaust gas to per kg H₂S bioreactor output.

Since the majority of H₂ nutrient comes from the water shift reaction and subsequent conversion of CO to CO₂, the CO₂ requirements to maintain the pH of the bioreactor is approximately balanced.

### Example 11

### Equilibrium Between Hydrogen Sulfide, Hydrosulfide and Sulfide

The equilibrium between hydrogen sulfide (H₂S) and hydrosulfide (HS⁻) occurs at a pH at approximately 7. The equilibrium between hydrosulfide and sulfide (S²⁻) occurs at a pH at approximately 13.9 (Pourbaix, 1966 p 546). As such, the predominant dissolved species below pH 7 is H₂S. Between pH 7 and 13.9, HS⁻ is the predominant dissolved species. Above pH 13.9, S²⁻ is the predominant dissolved species.

In normal bioreactor operation the predominant dissolve species is HS⁻, and this is the form of sulfide in bioreactor discharge solution. Sulfide in the form of H₂S is continuously removed from the bioreactor by an inert carrier gas. The concentration remaining can be controlled by solution pH adjustment using CO₂. When H₂S gas only is required, bioreactor discharge solution can be further contacted with CO₂ to reduce the pH below 7 and further stripped of H₂S. This could be required in the example of CuS precipitation from an acid leach stream, where it is undesirable to increase the solution pH by the addition of any bioreactor discharge solution.

Alternatively, when bioreactor discharge solution is to be stored for future use of its sulfide content, its pH will be kept as high as possible, and may even be contacted with an alkaline reagent and additional H₂S from an off-gas stream to further increase pH and sulfide in the S²⁻ form for more stable storage.

### Example 12

### Uses of Hydrogen Sulfide, Hydrosulfide and Sulfide

H₂S, HS⁻ and S²⁻ can precipitate certain dissolved metal ions as metal sulfides. Examples of precipitation reactions with CuSO₄ with each species are as follows

CuSO₄+H₂S→CuS+H₂SO₄

CuSO₄+HS⁻→CuS+HSO₄⁻

CuSO₄⁺S²⁻→CuS+SO₄²⁻

### Example 13

### Use of Thiosulfate from a Waste Water Source

For some applications there may be readily available alternatives to sulfur dioxide as the source of reduced sulfur for the bioreactor. Thiosulfate is a common contaminant in waste waters from petrochemical refining, and other chemical industries and it is also increasingly used as a reagent in hydrometallurgical processes. This is a favourable source of sulfur with a relatively low energy demand for reduction:

4H₂(g)+S₂O₃²⁻(aq)→2H₂S(g)+H₂O(l)+2OH⁻(aq)

This application combines the production of H₂S with the removal of a contaminant as well as the regeneration of an alkaline reagent. Thiosulfate is unstable in acid form and is generally present as the sodium salt so with pH control by CO₂ addition the bioreactor will generate a sodium carbonate/bicarbonate by-product which may be recovered from the discharge solution.

### Example 14

### Use of Dithionate from a Waste Water Source

The process may also be adapted to other reduced sulfur compounds that may be found in industrial wastewater streams. For example, the hydrometallurgical processing of manganese ores using sulfur dioxide may result in a waste stream containing dithionate and sulfite ions which requires disposal. Dithionate reduction proceeds according to the following balance:

7H₂(g)+S₂O₆²⁻(aq)→2H₂S(g)+4H₂O(l)+2OH⁻(aq)

Thus the energy source (H₂) requirement is only 12.5% less than sulfate reduction (3.5 H₂ versus 4H₂ per H₂S generated) but dithionate removal is also an important process requirement. Reduction of these compounds to treat the waste stream would produce H₂S which could be burned to regenerate sulfur dioxide reagent and to recover part of the energy put into the treatment.

1. A process for the biologically-catalyzed production of sulfide species including sulfide, hydrosulfide or hydrogen sulfide alone or in combination, utilizing sulfur dioxide containing combustion flue gas alone or in combination with non-sulfur dioxide containing flue gas, that in composite contain useable quantities of one or more biological nutrients or energy sources including, but not limited to carbon monoxide, hydrogen, carbon dioxide, nitrous oxide, nitric oxide, nitrogen dioxide, ammonia, carbonyl sulfide and fly ash.

2. The process in claim 1 where one or more gas streams fed to the bioreactor is at a temperature above ambient temperature and at least a portion of the heat in said gas stream is used to provide heat to the bioreactor.

3. The process in claim 2 where heat from said gas stream is used to maintain the bioreactor temperature in the range of 25-35° C.

4. The process in claim 1 where one or more of the gas streams are produced through the combustion of a suitable fuel source under controlled conditions of excess air.

5. The process in claim 4 where the fuel source is coal.

6. The process in claim 4 where the fuel source is a hydrocarbon fuel, which may include, but is not limited to one or more of methane, ethane, propane, butane, gasoline, diesel, kerosene, fuel oil or bunker C oil.

7. The process in claim 4 where the fuel source is petroleum coke.

8. The process in claim 4 where the fuel source is organic biomass such as wood or agricultural waste.

9. The process in claim 4 where the fuel source is a mineral concentrate containing at least one metallic sulfide mineral or elemental sulfur or a mixture of metallic sulfide minerals and elemental sulfur.

10. The process in claim 1 where one or more of the gas streams are produced through the combustion of a suitable fuel source under controlled conditions at or near a stoichiometric rate of air addition.

11. The process in claim 10 where the fuel source is coal.

12. The process in claim 10 where the fuel source is a hydrocarbon fuel, which may include, but is not limited to one or more of methane, ethane, propane, butane, gasoline, diesel, kerosene, fuel oil or bunker C oil.

13. The process in claim 10 where the fuel source is petroleum coke.

14. The process in claim 10 where the fuel source is organic biomass such as wood or agricultural waste.

15. The process in claim 10 where the fuel source is a mineral concentrate containing at least one metallic sulfide mineral or elemental sulfur or a mixture of metallic sulfide minerals and elemental sulfur.

16. The process in claim 1 where one or more of the gas streams are produced through the combustion of a suitable fuel source under controlled conditions with a rate of air addition below the stoichiometric requirement for complete combustion.

17. The process in claim 16 where the fuel source is coal.

18. The process in claim 16 where the fuel source is a hydrocarbon fuel, which may include, but is not limited to one or more of methane, ethane, propane, butane, gasoline, diesel, kerosene, fuel oil or bunker C oil.

19. The process in claim 16 where the fuel source is petroleum coke.

20. The process in claim 16 where the fuel source is organic biomass such as wood or agricultural waste.

21. The process in claim 16 where the fuel source is a mineral concentrate containing at least one metallic sulfide mineral or elemental sulfur or a mixture of metallic sulfide minerals and elemental sulfur.

22. The process in claim 1 where one or more of the gas streams is produced from a carbon-based fuel in a gasifier which uses a water-shift reaction to convert some or all of the carbon monoxide produced to hydrogen and carbon dioxide.

23. The process in claim 1 where one or more of the gas streams is produced from reaction of a carbon-based fuel with the temperature, pressure, air-fuel ratio and steam addition chosen to maximize the hydrogen content of the resulting gas stream.

24. The process in claim 1 where the bioreactor is operated under conditions above atmospheric pressure via control of gas inlet and outlet pressure.

25. The process in claim 1 where the bioreactor solution is maintained at a pH below 9.0 through the use of carbon dioxide contained in at least one of the gas streams fed to the bioreactor.

26. The process in claim 1 where the bioreactor solution is maintained at a pH above 6.0 through the adjustment of the addition of at least one of carbon dioxide and sulfur dioxide contained in at least one of the gas streams fed to the bioreactor.

27. The process in claim 1 where the bioreactor solution is maintained at a temperature above 20° C. by the operation of combustion and energy recovery stages in a manner that results in at least one of the gas streams feeding the bioreactor at a volume and temperature sufficient to allow the heat transfer required to maintain solution temperature at or above this level.

28. The process in claim 1 where the bioreactor solution is maintained at a temperature below 45° C. by the operation of combustion and energy recovery stages in a manner that results in all gas streams feeding the bioreactor being sufficiently reduced in temperature prior to reaching the bioreactor that the resulting heat transfer will not cause solution temperature to exceed this level.

29. The process in claim 51 where high temperature gases exhausted from a combustion stage are cooled using a steam generating boiler.

30. The process in claim 51 where high temperature gases exhausted from a combustion stage are cooled using a gas turbine.

31. The process in claim 51 where high temperature gases exhausted from a combustion stage are cooled using a heat exchanger.

32. The process in claim 51 where high temperature gases exhausted from a combustion stage are cooled using a heat pump.

33. The process in claim 1 where a portion of the hydrogen sulfide generated in the bioreactor is removed from the bioreactor as a gas in an off-gas stream containing hydrogen sulfide together with one or more of nitrogen, hydrogen, carbon monoxide, methane, carbon dioxide, water vapor and argon.

34. The process in claim 33 where the hydrogen sulfide-containing off-gas stream removed from the bioreactor is contacted with a waste water stream containing dissolved metals in one or more stages, resulting in the precipitation of insoluble metal sulfide compounds.

35. The process in claim 34 where all or a portion of the precipitated insoluble metal sulfide compounds are collected and dewatered, constituting a fuel source for a combustion stage.

36. The process in claim 35 where the exhaust gas from the sulfide mineral combustion is fed to the bioreactor to provide one or more of reduced sulfur compounds, heat, a carbon source, an energy source and biological nutrients.

37. The process in claim 33 where the hydrogen sulfide-containing off-gas stream removed from the bioreactor is contacted with a metallurgical process stream containing one or more dissolved metals, resulting in the formation of insoluble metal sulfide compounds.

38. The process in claim 37 where all or a portion of the precipitated insoluble metal sulfide compounds are collected and dewatered, constituting a fuel source for a combustion stage.

39. The process in claim 38 where the exhaust gas from the sulfide mineral combustion is fed to the bioreactor to provide one or more of reduced sulfur compounds, heat, an energy source and biological nutrients.

40. The process in claim 33 where the hydrogen sulfide-containing off-gas stream removed from the bioreactor is contacted with an alkaline solution to produce an alkali metal or alkali earth metal sulfide compound or compounds.

41. The process in claim 33 where the hydrogen sulfide-containing off-gas stream removed from the bioreactor is contacted with a mineral or industrial process stream to modify the solid surfaces or the solution chemistry.

42. The process in claim 34, where the bioreactor off-gas stream, following removal of all or part of the hydrogen sulfide, is transported to at least one fuel combustion stage where it is added as a supplement to the primary fuel.

43. The process in claim 33 where the hydrogen-sulfide containing off-gas stream removed from the bioreactor is burned under controlled conditions to produce a sulfur dioxide-containing stream for use in an industrial or mineral process.

44. The process in claim 1 where the bioreactor contains a mixed population of bacteria which may include a range of competing and complimentary species and which includes at least one species of sulfate reducing bacteria.

45. The process in claim 43 where the species of sulfate reducing bacteria present include at least one of desulfovibrio sp., desulfotomaculum sp., desulfobulbus sp., desulfobacter sp., desulfobacterium sp., desulfococcus sp., desulfonema sp., desulfosarcina sp., desulforhabdus sp., and campylobacter sp.

46. The process in claim 43 where one or more subspecies of bacteria present contain the enzyme nitrite reductase in their cell structure. This group may include, but is not limited to subspecies of desulfovibrio vulgaris, desulfovibrio desulfuricans or desulfobulbus propionicus.

47. A process for the biologically-catalyzed production of sulfide species including sulfide, hydrosulfide or hydrogen sulfide alone or in combination, utilizing divalent sulfur containing liquor in combination with nutrients.

48. A process as in claim 47 in which the divalent sulfur species is thiosulfate.

49. A process for the biologically-catalyzed production of sulfide species including sulfide, hydrosulfide or hydrogen sulfide alone or in combination, utilizing pentavalent sulfur containing liquor in combination with nutrients.

50. A process as in claim 47 in which the pentavalent sulfur species is dithionate.

51. The process as in claim 1, wherein the bioreactor solution is maintained at a temperature above 20° C. by the operation of combustion and energy recovery stages in a manner that results in at least one of the gas streams feeding the bioreactor at a volume and temperature sufficient to allow the heat transfer required to maintain the solution temperature at or above this level, and further wherein the bioreactor solution is maintained at a temperature below 45° C. by the operation of combustion and energy recovery stages in a manner that results in all gas streams feeding the bioreactor being sufficiently reduced in temperature prior to reaching the bioreactor that the resulting heat transfer will not cause solution temperature to exceed this level.

52. The process as in claim 37, wherein the bioreactor off-gas stream, following removal of all or a part of the hydrogen sulfide, is transported to at least one field combustion stage where is added the supplement to the primary fuel.

53. The process as in claim 40, wherein the bioreactor off-gas stream, following removal of all or a part of the hydrogen sulfide, is transported to at least one field combustion stage where is added the supplement to the primary fuel.

54. The process as in claim 41, wherein the bioreactor off-gas stream, following removal of all or a part of the hydrogen sulfide, is transported to at least one field combustion stage where is added the supplement to the primary fuel.
